(19) United States
(12) Patent Application Publication
(10) Pub. No.: US 2005/0271709 A1
(43) Pub. Date: Dec. 8, 2005

(54) Coated soy product and method for coating

(76) Inventors: Sean Mark Dazliel, Wilmington, DE (US); Thomas Friedman, Hockessin, DE (US); George A. Schurr, Newark, DE (US)

Correspondence Address: Lynne M Christenbury;E I du Pont de Nemours & Company, Legal Patents, Wilmington, DE, 19898, US

(21) Appl. No.: 10/524,673

(22) PCT Filed: Aug. 14, 2003
(86) PCT No.: PCT/US03/25884

§ 371 Date: Feb. 11, 2005

Related U.S. Application Data
(60) Provisional application No. 60/403,597, filed on Aug. 14, 2002.

(51) Int. Cl. A61K 35/78; A23K 1/165; A23K 1/17

(52) U.S. Cl. 424/442; 424/757; 426/635

(57) ABSTRACT

A process for coating a soy product or flour is described in which the moisture level of the coated soy product is substantially the same as the moisture level of the uncoated soy product. Coated soy products or coated flours made using the process of the invention are also disclosed.

This application claims the benefit of U.S. Provisional Application No. 60/403597, filed Aug. 14, 2002.

### FIELD OF THE INVENTION

This invention is in the field of coating particles and, in particular coating a soy product or flour.

### BACKGROUND OF THE INVENTION

The use of coating technology in the food industry has been growing rapidly. It involves the coating or entrapment of a pure material or mixture into another material. The coated or entrapped material is usually a liquid but can be a solid or gas. The number of coated food products has increased significantly with the introduction of more cost effective preparation techniques and materials. However, there exists a need to reduce production costs and increase the number of food-grade materials as well as addressing problems relating to stability, dispersability, weftability, controlled release, and delivery of food ingredients.

Many benefits are offered to the food industry by coating technology. For example, such technology can improve the flavor, aroma, stability, appearance, nutritional value and texture of food products. It can also make food ingredients easier to handle, easier to solubilize, offer protection from moisture, heat or other extreme conditions and increase stability against oxidation. It is important to ensure nutritive value and sensory quality at least equal to the original food. It is also important, in food applications, to have a safe, non-toxic, edible coating material.

Nutraceutical ingredients and products which target specific health problems can reduce off-flavors contributed by certain vitamins and minerals, permit time-release of the nutrients, enhance stability to extremes of temperature and moisture, and reduce undesirable chemical interactions with other ingredients. Color degradation, rancidity, water absorption and yeast growth can be controlled. Flowability can be improved and clumping and caking can be reduced. The texture and appearance of bakery products can be improved by using coated leavening agents. Nutraceutical snacks, such as soy-based products, may also use coating to protect flavor during extrusion or improve dispersibility or weftability.

People who frequently consume soy foods have lower rates of many types of cancer, including breast, colon, lung and prostate cancers. They also have much lower rates of heart disease. Studies have shown that adding soy protein to the diet can dramatically lower cholesterol levels and significantly reduce the risk for heart disease. As a part of a healthy diet, soy foods also can help control diabetes and kidney disease, and may reduce the risk for osteoporosis.

However, there is a need for improved coating technology to improve delivery and protection of ingredients in food applications while addressing cost concerns.

Microencapsulation has been defined as a process by which small particles (generally between 1 to 1000 microns in diameter) of solid, liquid or gas are packaged within a secondary material to form a microcapsule. (Sanguansri et al., Microencapsulation for Innovative Ingredients A Scoping Study: Opportunities for Research into the Microencapsulation of Food Ingredients, Food Science Australia, May 2001).

Gibbs et al., Intemational Joumal of Food Sciences and Nutrition, 50:213-224 (1999), discusses a review of encapsulation in the food industry.

WO 93/07761, published on Apr. 29, 1993, describes a dry microparticulated protein product which may be used as a fat substitute when rehydrated.

WO 94/08468, published on Apr. 28, 1994, describes a free-flowing spray-dried powder product containing a food-improving surface-active substance selected from organic esters of lipid nature such as whipping or aerating emulsifiers which are glycerol or polyglycerol partial esters with edible fatty acids.

An apparatus and process for coating small solid particles, such as powdery or granular materials, are described in WO 97/07879 published Mar. 6, 1997, and assigned to E.I. du Pont de Nemours and Company. This process involves metering a liquid composition comprising a coating material, where the liquid composition is either a solution, slurry or melt, into a flow restrictor and injecting a gas stream through the flow restrictor concurrently with the metering of the liquid composition to create a zone of turbulence at the outlet of the flow restrictor, thereby atomizing the liquid composition. The gas stream is heated prior to injecting it through the flow restrictor. A solid particle is added to the zone of turbulence concurrently with the metering of the liquid composition and the injection of the heated gas to mix the solid particle with the atomized liquid composition. The mixing at the zone of turbulence coats the solid particle with the coating material.

WO 97/07676 to E.I. du Pont de Nemours and Company discloses the apparatus of WO 97/07879, along with the use of the apparatus in a process for coating crop protection solid particles. Coatings are water-insoluble, and coating thicknesses are represented by weight percent rather than thickness.

U.S. Pat. No. 6,015,773, issued to Wysong et al. on Jan. 18, 2000, describes a crop protection composition comprising a mononucleate crop protection solid particle coated with water-insoluble coating material having a diameter in the range from 0.5 to 50 micrometers. This composition is made by a process which results in substantial non-agglomeration of the coated particles.

Applicants' assignee's copending application having application Ser. No. 10/174,687, filed Jun. 19, 2002, and having Attorney Docket Number BB-1879 US NA discloses a process for dry coating a food particle having its largest diameter in the range from 0.5 mm to 20.0 mm with a liquid coating material. The coated food particle has a moisture level that is substantially the same as the moisture level of the uncoated food particle. Also disclosed is a process for encapsulating a frozen liquid particle having a size in the range from 5 micrometers to 5 millimeters with a liquid coating material.

Applicants' assignees' copending, concurrently filed herewith provisional applications having Attomey Docket numbers CL2101, CL2148, CL2150, CL2178 and PTI sp1255 disclose subject matter related to the present application, and are specifically incorporated herein by reference.

U.S. Pat. Nos. 3,241,520 and 3,253,944 disclose a particle coating method wherein relatively large pellets, granules and particles are suspended in a stream of air while coating material in a liquid form is mixed with the particles.

U.S. Pat. No. 6,224,939 B1 issued to Cherukuri et al May 1, 2002, describes a method and apparatus for the encapsulation of feedstock, wherein a solid matrix additive is spray injected in a free-flow condition.

Shahidi et al., Critical Reviews in Food Science and Nutrition, 33(6):501-547 (1993) presents a review of the art of microencapsulation of food ingredients.

### SUMMARY OF THE INVENTION

The present invention includes a process for coating a soy product, the process comprising the steps of:

(a) metering a liquid coating material through a flow restrictor,

(b) injecting a gas stream through the flow restrictor concurrently with step (a) to (i) atomize the liquid coating material and (ii) create turbulent flow of the gas stream and the atomized liquid coating material, wherein the gas stream is optionally heated; and

(c) adding a soy product to the turbulent flow region concurrently with steps (a) and (b), wherein the soy product mixes with the atomized liquid coating material to provide a coated soy product.

In a second embodiment, this invention includes repeating steps

(a)-(c) at least once wherein the liquid coating material is the same or different.

This process can be practiced with any soy product such as soy protein isolate, soy concentrate, soy meal, soy cotyledon fiber, dehulled soybeans, soy hypocotyls, soy grits, soy chips, soy flour, textured soy protein, and soy flakes.

In another aspect, the liquid coating material is selected from the group consisting of a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, an emulsification agent or a mixture thereof.

In still another aspect, this invention includes a coated soy product made by the process of the invention, use of such coated soy product in food applications as well as a food, nutritional supplement, beverage, infant formula, pet food and animal feed comprising a coated soy product made by the process of the invention.

In a third embodiment, this invention includes a process for coating a flour, the process comprising the steps of:

(a) metering a liquid coating material through a flow restrictor;

(b) injecting a gas stream through the flow restrictor concurrently with step (a) to (i) atomize the liquid coating material and (ii) create turbulent flow of the gas stream and the atomized liquid coating material, wherein the gas stream is optionally heated; and

(c) adding a flour to the turbulent flow region concurrently with steps (a) and (b), wherein the flour mixes with the atomized liquid coating material to provide a coated flour.

In another aspect, the flour is selected from the group consisting of soy flour, wheat flour, oat flour, rye flour, barley flour, rice flour, millet flour and corn flour.

In still another aspect, the liquid coating material is selected from the group consisting of a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, an emulsification agent or a mixture thereof.

In a still further aspect, this invention includes further comprising repeating steps (a)-(c) at least once wherein the liquid coating material is the same or different.

Also of interest is a coated flour made by the process of the invention as well as any food, baked good, snack food comprising such coated flour made by the process of the invention.

### BRIEF DESCRIPTION OF THE FIGURES

FIG. 1 depicts soy protein processing.

FIG. 2 is a schematic diagram of a portion of the apparatus in accordance with the present invention.

FIG. 3 is a cut away, expanded, cross-sectional view of a portion of the apparatus show in FIG. 2.

FIG. 4 depicts an alternate configuration of the apparatus shown in FIGS. 2 and 3.

### DETAILED DESCRIPTION OF THE INVENTION

All patents, patent applications and publications referred to herein are incorporated by reference in their entirety.

In the context of this disclosure, a number of terms shall be utilized.

The term “soy product” as used herein refers to any product derived from the processing of a soybean.

The term “flour” as used herein refers to finely-ground meals, and includes both food flours and non-food flours.

The term “coating” as used herein refers to adherence, adsorption, loading and/or incorporation, to some extent, of at least one liquid coating material onto and/or into a particle or particles. The coating may be of any thickness; it is not necessarily uniform, nor is the entire surface necessarily coated. The term “dry coating” as used herein refers to a coating process wherein the particle to be coated is coated in its dry form, the process does not require dispersing the particles in a continuous liquid phase prior to coating, and at the conclusion of the process the particle has no substantial gain in moisture level relative to its uncoated form. The terms “coating” and “dry coating” are used interchangeably herein. As used herein, the term coating does not necessarily imply that the coated particle has been protected from oxidation or diffusion of volatile materials through the coating.

The term “moisture level” as used herein refers to the amount of moisture, for example water or solvent, that is present in the food particles before or after coating.

The term “oxidation” as used herein refers to the process wherein the atoms in an element lose electrons thereby making it more electropositive. The valence of the element is correspondingly increased resulting in destruction of fat soluble vitamins, loss of natural colors, decrease or change in aroma and flavor, and creation of toxic metabolites.

The term “size” as used herein refers to the longest diameter or longest axis of the particle being coated.

The term “volatile” as used herein refers to a compound or material that is readily vaporizable at a relatively low temperature, i.e., it evaporates rapidly. “Volatiles” may refer, for example, to the aroma volatiles within foods, to volatiles in the environment that may diffuse into foods and cause an “off” taste or smell, or to water moisture in gaseous form.

The present invention includes a process for coating a soy product, the process comprising the steps of:

(a) metering a liquid coating material through a flow restrictor;

(b) injecting a gas stream through the flow restrictor concurrently with step (a) to (i) atomize the liquid coating material and (ii) create turbulent flow of the gas stream and the atomized liquid coating, wherein the gas stream is optionally heated; and

(c) adding a soy product to the region of turbulent flow concurrently with steps (a) and (b), wherein the soy product mixes with the atomized liquid coating material in the region of turbulent flow to provide a coated soy product, Thus, unlike a fluidized bed apparatus in which the particles to be coated undergo batchwise successive coating within the bed to ensure a prolonged residence time in the treating vessel in order to obtain adequate coating, the process of the invention is practiced without the need for such batchwise successive coating. Indeed, the process of the invention can be considered as a substantially “one pass” process with an extremely short residence time in the region in which coating occurs.

In another aspect, the above-escribed process further comprises repeating steps (a)-(c) at least once wherein the liquid coating material is the same or different.

Thus, coated soy product can be coated with a combination of liquid coating materials to enhance dispersibility, wettability, oxidative stability and increase shelf life. In addition, unique combinations of flavors, colors, aromas, etc. could be coated onto the particles. Multiple coatings thus applied can lead to uniquely tailored coated soy product with desired colors, flavorings and freshness aspects; each coating having the ability to retain its original integrity and function, in that there is minimal “mixing” of subsequent layers which are applied to the particles.

Additionally, such soy products can be further coated multiple times with the same liquid coating material, enabling the claimed process to yield soy product having particularly controlled thickness of the coating material. Such soy products that are coated multiple times with the same liquid coating material can be coated in a continuous process. It is also possible to provide multiple coatings to a particle by delivering the output of a first apparatus to the feed of a second apparatus in a continuous process.

There are several benefits of the instant process. It is believed that the process of the instant invention is more cost efficient than currently conducted coating processes, which commonly depend upon spray drying techniques. Further, in one particularly important aspect, the instant process has the flexibility to be operated as a continuous process. Further, overall particle quality appears to be improved since this is a dry coating process, wherein the liquid coating and drying step occur during the same pass of the soy product through the apparatus of the invention. Overall particle quality of the soy product is also improved in that the particles that have been coated with the instant process have been observed to retain their morphology, structural integrity and particle size throughout the process. And importantly, the starting moisture level of the coated particles is substantially unchanged during the process. In other words, the moisture level of the coated soy product will be substantially the same as the moisture level of the uncoated soy product. It is desirable that the process yields final coated soy product that have not lost moisture and appear too dry, or have taken on additional moisture and become damp, soggy or agglomerated.

The flexibility which is inherent in the operation of the apparatus and process of the invention can result in production of high quality coated soy product having carefully-controlled and unique characteristics. For example, concentration values of the coating liquid, flow rates of the solid particle feed and the liquid coating feed, ratios of liquid feeds to solid feeds, and temperature and velocity of the gas streams can all be easily varied to yield such coated soy product particles with particular desired characteristics.

The size of the coated soy product should not exceed 20.0 mm. The lower limit on size will depend on the soy product being coated, intended use of the product, storage conditions, type of liquid coating material, etc.

Suitable liquid coating materials will be those which can be used in any food application such as any food, nutritional supplement, beverage, infant formula and the like. Applications intended for human consumption should generally utilize materials that are generally recognized as safe (“GRAS”). If the intended application is for incorporation into a pet food or animal feed, then other liquid coating materials may be suitable.

For example, some materials recognized as GRAS include but are not limited to the following: polysaccharides/hydrocolloids such as starch, agar/agarose, pectin/polypectate, carrageenan and other gums; proteins such as gelatin, casein, zein, soy and albumin; fats and fatty acids such as mono- , di-, and triglycerides, lauric, capric, palmitic and stearic acid and their salts; cellulosic derivatives; hydrophilic and lipophilic waxes such as shellac, polyethylene glycol, camauba wax or beeswax; sugar derivatives, etc.

Examples of such liquid coating materials include, but are not limited to, a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, an emulsification agent or a mixture thereof.

Examples of sweetening agents include, but are not limited to, sugar substitutes such as saccharin, cyclamate, monellin, thaumatins, curculin, miraculin, stevioside, phyllodulcin, glycyrrhizin, nitroanilines, dihydrochalcones, dulcin, suosan, guanidines, oximes, oxathiazinone dioxides, aspartame, alitame, and the like. There can also be mentioned monosaccharides and oligosaccharides. Examples of monosaccharides include, but are not limited to, galactose, fructose, glucose, sorbose, agatose, tagatose and xylose. As oligosaccharides there can be mentioned, sucrose, lactose, lactulose, maltose, isomaltose, maltulose, saccharose and trehalose. Other sweetening agents that can also be used include, but are not limited to, high fructose corn syrup or sugar alcohols.

Examples of food flavoring agents or enhancers include, but are not limited to, monosodium glutamate, maltol, 5′-mononucleotides, such as inosine, and the like.

Examples of food colors include, but are not limited to, tartrazine, riboflavin, curcumin, zeaxanthin, β-carotene, bixin, lycopene, canthaxanthin, astaxanthin, β-apo-8′-carotenal, carmoisine, amaranth, Ponceau 4R (E124), Carmine (E120), anthocyanidin, erythrosine, Red 2G, Indigo Carmine (E 132), Patent Blue V (E 131), Brilliant blue, chlorophyll, chlorophyllin copper complex, Green S (E142), Black BN (E151), and the like.

Examples of food aroma agents include, but are not limited to, carbonyl compounds, pyranones, furanones, thiols, thioethers, di- and trisulfides, thiophenes, thiazoles, pyrroles, pyridines, pyrazines, phenols, alcohols, hydrocarbons, esters, lactones, terpenes, volatile sulfur compounds and the like.

Examples of an anti-caking agents include, but are not limited to, sodium, potassium, calcium hexacyanoferrate (II), calcium silicate, magnesium silicate, tricalcium phosphate, magnesium carbonate and the like.

Examples of humectants include, but are not limited to, 1,2-propanediol, glycerol, manitol, sorbitol and the like.

Examples of antimicrobial agents include, but are not limited to, benzoic acid, PHB esters, sorbic acid, propionic acid, acetic acid, sodium sulfite and sodium metabisulfite, diethyl pyrocarbonate, ethylene oxide, propylene oxide, nitrite, nitrate, antibiotics, diphenyl, o-phenylphenol, thiabendazole and the like.

Examples of antioxidant agents include, but are not limited to, tocopherols, 2,6-di-tert-butyl-p-cresol (BHT), tert-butyl-4-hydroxyanisole (BHA), propylgallate, octylgallate, dodecylgallate, ethoxyquin, ascorbyl palmitate, ascorbic acid and the like.

Examples of surface modifying agents include, but are not limited to, mono-, diaglycerides and derivatives, sugar esters, sorbitan fatty acid esters, polyoxyethylene sorbitan esters, stearyl-2-lactylate and the like.

Examples of nutritional supplementing agents include, but are not limited to, vitamins group consisting of fat soluble vitamins group consisting of retinol (vit A), calciferol (vit D), tocopherol (vit E), phytomenadione (vit K1), water soluble vitamins group consisting of thiamine (vit B1), riboflavin (vit B2), pyridoxine (vit B6), nicotinamide (niacin), pantothenic acid, biotin, folic acid, cyanocobalamin (vit B12), ascorbic acid (vit C), polyunsaturated fatty acids (PUFA), and the like.

Other carbohydrates which can be used in a liquid coating material include polysaccharides such as agar, alginates, carrageenans, furcellaran, gum arabic, gum ghafti, gum tragacanth, karaya gum, guaran gum, locust bean gum, tamarind flour, arabinogalactan, pectin, starch, modified starches, dextrins, cellulose, cellulose derivatives, hemicelluloses, xanthan gum, scleroglucan, dextran, polyvinyl pyrrolidone and the like.

Examples of lipids include, but are not limited to, saturated and unsaturated fatty acids, mono- and diacylglycerols triacylglycerols, phospholipids, glycolipids, phosphatidyl derivatives, glycerolglycolipids, sphingolipids, lipoproteins, diol lipids, waxes, cutin and the like.

Examples of minerals include, but are not limited to, salts of sodium, potassium, magnesium, calcium, chloride, phosphate, iron, copper, zinc, manganese, cobalt, vanadium, chromium, selenium, molybdenum, nickel, boron, silica, silicon, fluorine, iodine, arsenic and the like.

The process of the invention can be practiced with any soy product derived from the processing of soybeans. Soy products include, but are not limited to soy protein products. There are three major groups of soy protein products. These groups are based on protein content, and range from 40% to over 90%. All three basic soy protein product groups (except full fat flours) are derived from defatted flakes. They are: soy flours and grits, soy protein concentrates and soy protein isolates. Other soy products derived from the processing of soybeans include soy fiber, in particular, soy cotyledon fiber.

There are also specialty products based on traditional Oriental processes, which utilize the entire bean as starting material.

For example, a soy product can be selected from the group consisting of soy protein isolate, soy concentrate, soy meal, soy cotyledon fiber, dehulled soybeans, soy hypocotyls, soy grits, soy chips, soy flour, textured soy protein, and soy flakes and the like. Additional examples are provided in Table 1.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Milk
Soy Protein Isolates
Soy Protein Concentrates
Textured Soy Proteins
Textured Flours and Concentrates
Textured Concentrates
Textured Isolates
^{a}See Soy Protein Products: Characteristics, Nutritional Aspects and Utilization (1987). Soy Protein Council

“Processing” refers to any physical and chemical methods used to obtain the products listed in Table 1 and includes, but is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [Poult. Sci. 69:76-83 (1990)]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

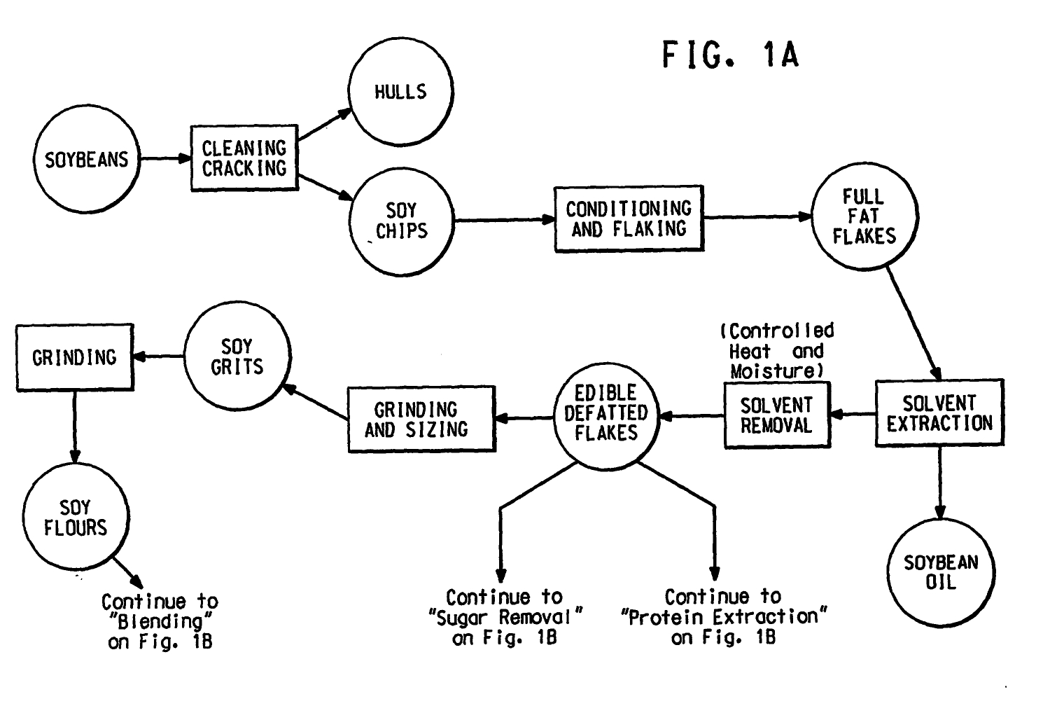

Soy processing is depicted in FIG. 1.

“Soy cotyledon fiber material”, as used herein, is defined as the fraction of dehulled, defatted, and degermed soybeans that is insoluble in an aqueous solution having a pH substantially above or below the isoelectric point of the combined 7S and 11S fractions of soy protein (typically a pH of 6.0 or greater, or 3.0 or less, where the isoelectric point of the 7S fraction of soy protein is 4.5 and the isoelectric point of the 11S fraction of soy protein is 5.3). Soy cotyledon fiber material, as used herein, includes pure soy polysaccharide fiber—both soluble and insoluble fiber fractions—but also includes compositions containing soy polysaccharide fiber in combination with soy protein and other minor constituents such as ash and fat. For example, Fibrim 1450, a commercially available soy cotyledon fiber material available from DuPont Protein Technologies Inc., contains by weight: 80.6% dietary fiber (as is); 12.2% protein (N×6.25%, as is); 3.6% ash, 0.9% fat (acid hydrolysis); and other minor constituents. “Soy cotyledon fiber material”, as used herein, does not include soy hull fiber.

Soy cotyledon fiber materials useful in the present invention may be produced from commercially available soy flour, soy grits, soy meal, or soy flakes. The soy flour, soy grits, soy meal, or soy flakes is/are extracted with an aqueous solution having a pH substantially above or below the isoelectric point of soy protein (pH 4.5) to extract protein and water soluble carbohydrates from the cotyledon fiber. Preferably the aqueous extractant has a pH of above pH 6.0 or below pH 3.0. Most preferably the aqueous extractant is an aqueous alkaline solution having a pH of from 8.0 to 10.0, preferably an aqueous sodium hydroxide solution. Alternatively the preferred aqueous extractant is an acidic solution having a pH of from 1.0 to 3.0, preferably a hydrochloric acid solution.

After extracting the protein and water soluble carbohydrates from the cotyledon fiber material, the liquid extract containing the protein and carbohydrates is separated from the fiber material. The extract may be separated from the fiber material in accordance with conventional separation techniques such as centrifugation and filtration.

Soy cotyledon fiber materials that are useful in the present invention are available commercially. For example, FIBRIM® 1260 and FIBRIM® 1450, available from DuPont Protein Technologies, are soy cotyledon fiber materials that are useful in the present invention.

A coated soy product made according to the process of the present invention can be incorporated into a wide variety of food and beverage applications. For example, there can be mentioned meats such as ground meats, emulsified meats, marinated meats, and meats injected with an coated soy product of the invention; nutritional supplements; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise; and chip dips; and food bars.

There also can be mentioned, a cereal food product, a snack food product, a baked good product, a fried food product, a health food product, infant formula, beverages, a nutritional supplement, a dairy product, a pet food product, or animal feed.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to, whole grain, crushed grain, grits, flour, bran, germ, breakfast cereals, extruded foods, pastas, and the like.

A baked good product comprises any of the cereal food products mentioned above and has been baked or processed in a manner comparable to baking, i.e., to dry or harden by subjecting to heat. Examples of a baked good product include, but are not limited to bread crumbs, baked snacks, mini-biscuits, mini-crackers, mini-cookies, and mini-pretzels.

A snack food product comprises any of the above or below described food products.

A fried food product comprises any of the above or below described food products that has been fried.

A health food product is any food product that imparts a health benefit. Many oilseed-derived food products may be considered as health foods. There can be mentioned soybeans, flax seed, sesame seed, pumpkin seeds, sunflower seeds, or food products processed from these seeds or which are incorporated into foods. For example, soy nuggets and soy nuts can be mentioned. In addition to oilseed-derived food products, fruit-derived food products can be mentioned such as fruit bits, dried berries, and the like.

A beverage is any drinkable liquid. For example, there can be mentioned non-carbonated drinks; carbonated drinks; fruit juices, fresh, frozen, canned or concentrate; still or sparkling water; flavored or plain milk drinks, etc. Adult and infant nutritional formulas are well known in the art and commercially available (e.g., Similac®, Ensure®, Jevity®, and Alimentum® from Ross Products Division, Abbott Laboratories).

Infant formulas are liquids or reconstituted powders fed to infants and young children. They serve as substitutes for human milk. Infant formulas have a special role to play in the diets of infants because they are often the only source of nutrients for infants. Although breast-feeding is still the best nourishment for infants, infant formula is a close enough second that babies not only survive but thrive. Infant formula is becoming more and more increasingly close to breast milk.

A dairy product is a product derived from milk. These products include, but are not limited to, whole milk, skim milk, fermented milk products such as yogurt or sour milk, cream, butter, condensed milk, dehydrated milk, coffee whitener, ice cream, cheese, whey products, lactose, etc.

A pet food product is a product intended to be fed to a pet such as a dog, cat, bird, reptile, fish, rodent and the like. These products can include the cereal and health food products above, as well as meat and meat byproducts, grass and hay products, including but not limited to alfalfa, timothy, oat or brome grass and the like.

Animal feed is a product intended to be fed to animals such as turkeys, chickens, cattle and swine and the like. As with the pet foods above, these products can include cereal and health food products, meat and meat byproducts, and grass and hay products as listed above.

In another aspect, this invention includes any coated soy product made using the process of this invention as well as the use of a coated soy product made by the process of invention as a food ingredient, a nutritional supplement ingredient, a beverage ingredient, an infant formula ingredient, a pet food ingredient or an animal feed ingredient.

In still a further aspect, this invention includes a process for coating a flour, the process comprising the steps of:

(a) metering a liquid coating material through a flow restrictor;

(b) injecting a gas stream through the flow restrictor concurrently with step (a) to (i) atomize the liquid coating material and (ii) create turbulent flow of the gas stream and the atomized liquid coating material, wherein the gas stream is optionally heated; and

(c) adding a flour to the region of turbulent flow concurrently with steps (a) and (b), wherein the flour mixes with the atomized liquid coating material in the region of turbulent flow to provide a coated flour.

This process of the invention can be practiced with any flour be it a food flour or a non-food flour. Most flours have a particle size generally in the range: 10 microns to 1,000 microns. Most food flours are obtained from cereals. Cereals are a source of two major groups of hydrocolloids: starches and microcrystalline cellulose. (Hydrocolloids are polysaccharides: carbohydrate polymers of repeating sugar units). Microcrystalline cellulose is a non-fibrous form of cellulose. It disperses in water, but it is not soluble. For the most part, the plain, unmodified form of microcrystalline cellulose is used as a non-nutritive filler, binder and flow aid.

Examples of flours which can be coated using the process of the invention include, but are not limited to, soy flour, wheat flour, oat flour, rye flour, barley flour, rice flour, millet flour, corn flour, dietary fiber whether soluble or insoluble. Common sources of soluble fiber: barley, oats, apples, beans, citrus fruits, many vegetable, peas, psyllium seed, squash, etc. Common sources of insoluble fiber are corn, flaxseed, whole-wheat and whole grain products, etc. Grains that have been refined have had the fiber removed. White flour, for example, is whole-wheat flour that has had the fiber removed during refining.

Any of the liquid coating materials described above are suitable to coat flours as well.

Also, the process of the invention further comprises repeating steps (a)-(c) at least once wherein the liquid coating material is the same or different.

In another aspect, this invention includes any food comprising a coated flour made by the process of the invention. Examples of such foods include, but are not limited to baked goods, snack foods. Also, use of a coated flour made by the process of the invention as a food ingredient falls within the scope of the invention.

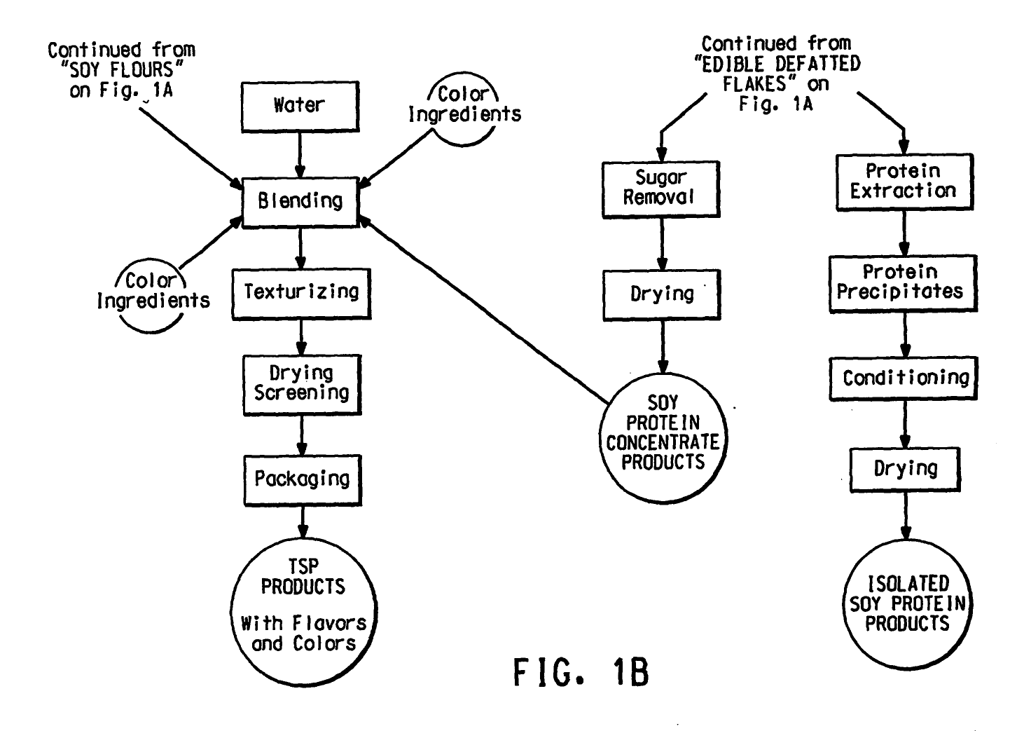

The apparatus used to practice the process of this invention is generally as described in commonly-owned PCT application WO 97/07879 which is discussed above. An apparatus according to the present invention is shown generally at 10 in FIG. 2.

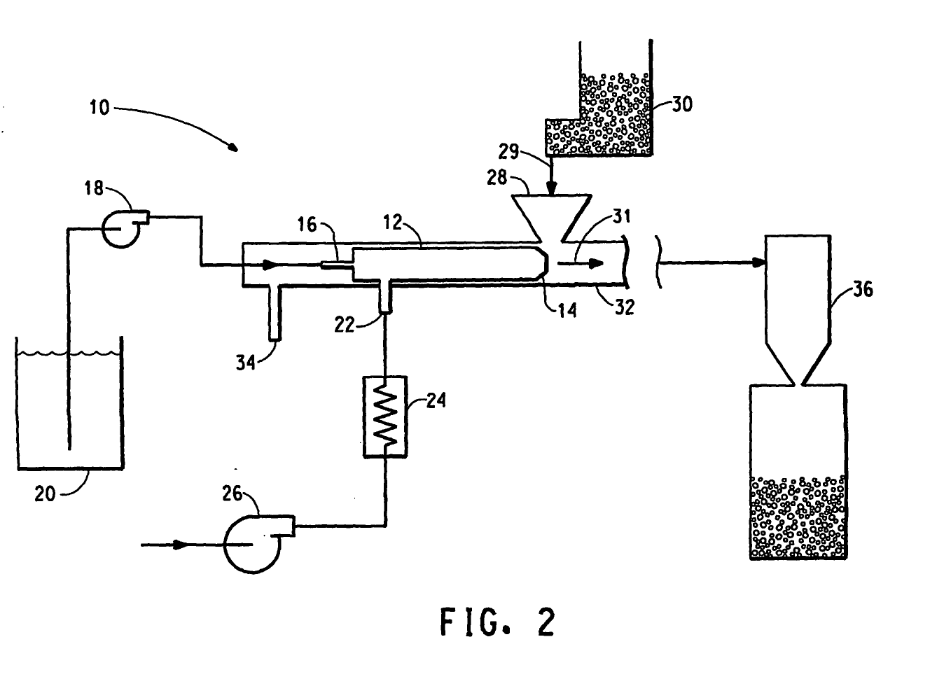

The apparatus of the present invention comprises a first chamber, shown at 12 in FIGS. 2 and 3. A flow restrictor 14 is disposed at one end of the first chamber. The flow restrictor is typically disposed at the downstream end of the first chamber, as shown in FIGS. 2 and 3. Flow restrictor 14 has an outlet end 14a, as shown in the detailed view of FIG. 3. Although the flow restrictor is shown as a different element from the first chamber, it may be formed integrally therewith, if desired. The flow restrictor of the present invention may have various configurations, as long as it serves to restrict flow and thereby increase the pressure of the fluid passing through it. Typically, the flow restrictor of the present invention is a nozzle.

A first, or liquid, inlet line 16 as shown in FIGS. 2 and 3 is disposed in fluid communication with the first chamber for metering a liquid composition into the chamber. Liquid inlet line 16 meters the liquid composition into first chamber 12 through the outlet of flow restrictor 14, and preferably in the center of the flow restrictor when viewed along the axial length thereof. The liquid composition is metered through liquid inlet line 16 by a metering pump 18 from a storage container 20 containing the liquid composition as shown in FIG. 2.

The liquid composition may be a solution, where a material which is used as the coating material is dissolved in a liquid, or a slurry, or an emulsion where a material which is used as the coating material is undissolved in a liquid. Alternatively, the liquid composition may be a melt, which is used as the coating material. By melt is meant any substance at a temperature at or above it melting point, but below its boiling point. In any of these cases, the liquid composition may include components other than the coating material. It should be noted that when the liquid composition is a melt, storage container 20 must be heated to a temperature above the melt temperature of the liquid composition in order to maintain the liquid composition in melt form.

The apparatus for coating a particle further includes a second, or gas, inlet line 22 disposed in fluid communication with the first chamber as shown in FIGS. 2 and 3. Generally, the gas inlet line should be disposed in fluid communication with the first chamber upstream of the flow restrictor. Gas inlet line 22 injects a first gas stream through the flow restrictor to create turbulent flow of the gas stream. The turbulence subjects the liquid composition to shear forces that atomize the liquid composition.

The first gas stream should have a stagnation pressure sufficient to accelerate the gas to at least one-half the velocity of sound, or greater, prior to entering the flow restrictor to ensure that a turbulent flow of gas of sufficient intensity will be formed at the outlet of the flow restrictor. The velocity of sound for a particular gas stream, e.g., air or nitrogen, will be dependent on the temperature of the gas stream. This is expressed by the equation for the speed of sound, C:
C=\sqrt{kgRT}

where:
k=ratio of specific heats for the gas
g=acceleration of gravity
R=universal gas constant
T=absolute temperature of the gas

Thus, the acceleration of the first gas stream is dependent on the temperature of the gas stream.

As noted above, it is the pressurized gas that causes the atomization of the liquid composition. The pressure of the liquid composition in the liquid inlet line just needs to be enough to overcome the system pressure of the gas stream. It is preferable that the liquid inlet line has an extended axial length upstream of the flow restrictor 14. If the liquid inlet line is too short, the flow restrictor becomes plugged.

The apparatus of the present invention also comprises means disposed in the second inlet line and upstream of the flow restrictor for optionally heating the first gas stream prior to injection through the flow restrictor. Preferably, the heating means comprises a heater 24 as shown in FIG. 2. Alternatively, the heating means may comprise a heat exchanger, a resistance heater, an electric heater, or any type of heating device. Heater 24 is disposed in second inlet line 22. A pump 26 as shown in FIG. 2 conveys the first gas stream through heater 24 and into first chamber 12. When a melt is used as the coating material, the gas stream should be heated to a temperature around the melt temperature of the liquid composition to ensure solidification of the melt on the particles. As also noted above for the apparatus, when using a melt, it is also helpful if auxiliary heat is provided to the first inlet line, which supplies the melt prior to injection, to prevent pluggage of the line.

The apparatus of the present invention further includes a second chamber 32 surrounding the first chamber as shown in FIGS. 2 and 3. The second chamber encloses the turbulent flow of gas. The apparatus of the present invention further includes a hopper 28 as shown in FIGS. 2 and 3. Hopper 28 introduces a particle into the region of the second chamber 32 in which turbulent flow of the gas has been created. It is preferable that the outlet end of the flow restrictor is positioned in the first chamber beneath the hopper at the centerline of the hopper (i.e., the region in which the turbulent flow of gas is created). This ensures that the particles are introduced directly into the turbulent flow of gas. This is important because, as noted above, the turbulence subjects the liquid composition to shear forces that atomize the liquid composition. It also increases operability by providing a configuration for feeding the particles most easily. In addition, the shear forces disperse and mix the atomized liquid composition with the particles, which allows the particles to be coated within the turbulent flow. Hopper 28 may be fed directly from a storage container 30 as shown by arrow 29 in FIG. 2. The hopper of the present invention may include a metering device for accurately metering the particles at a particular ratio to the liquid feed from liquid inlet line 16 into the zone of turbulence. This metering establishes the level of coating on the particle. Typically, the hopper of the present invention is open to the atmosphere. When a melt is used, it is preferred that the particles are at ambient temperature because this facilitates solidification of the melt after the melt which is initially at a higher temperature, coats the particle in the zone of turbulent flow.

The apparatus of the present invention further includes an inlet 34 for introducing a second gas stream into the second chamber. The inlet of the second gas stream is preferably positioned at or near the upstream end of second chamber 32. The outlet of second chamber 32 is connected to a collection container, such as that shown at 36 in FIG. 2. The second gas stream acts to reduce any tendency for batchwise successive coating within the region of turbulent flow and cools and conveys the coated particles toward the collection container as illustrated by arrow 31 in FIG. 3. In particular, when a solution or slurry is used, the solid of the solution or slurry cools between the zone of turbulence and container so that by the time the particle reaches the container, a solid coating comprising the solid of the solution or slurry is formed on the particle. When a melt is used, the liquid composition cools within the zone of turbulence so that by the time the particle reaches the container, a solid coating comprising the melt is formed on the particle. The first gas stream, as well as the second gas stream, is vented through the top of collection container 36.

For the configuration as shown in FIGS. 2 and 3, inlet 34 may be connected to a blower, not shown, which supplies the second gas stream to the second chamber. However, the blower and second chamber 32 may be eliminated, and the first gas stream may be used to cool the particles and to convey them to container 36. In this case, the solid from the solution or slurry or the melt cools/and solidifies on the particle in the atmosphere between the zone of turbulence and the collection container, and the coated particles fall into collection container 36.

It is preferable that the axial length of the region of the second chamber in which the turbulent flow is created is about ten times the diameter of the second chamber. This allows the pressure at the outlet of the flow restrictor to be at a minimum. Particles are fed into second chamber 32 as shown in FIGS. 2 and 3 near the outlet of the flow restrictor, which is preferably positioned at the centerline of the hopper. If the pressure at the outlet is too great, the particles will back flow into the hopper.

The pressure of the second gas stream must be sufficient to assist in conveying the coated particles from the zone of turbulence to the collection zone, but should be at lower than the pressure of the first gas stream. This is because a high relative velocity difference between the first gas stream and the second gas stream is needed to produce a sufficient degree of turbulence to coat the particles.

Further in accordance with the present invention, there is provided a process for dry coating a soy product or flour with a liquid coating material. The process provides a 1-step process, whereby materials to be coated are fed into the apparatus, coated, and collected without need of separation and/or filtration of the solids from liquids.

It should be noted that the process of the present invention may be practiced using the apparatus illustrated in FIGS. 2, 3 and 4, although it should be understood that the process of the present invention is not limited to the illustrated apparatus. Moreover, it should be noted that while one pass, or cycle, of the process of the present invention substantially or completely coats the particle, more than one pass may be used to adhere additional coating material to the particle, depending on the desired thickness of the liquid coating material.

The process comprises the steps of metering a liquid composition into a flow restrictor, such as flow restrictor 14 as shown in FIGS. 2 and 3. As described above for the apparatus, the liquid composition may be a solution, slurry, emulsion or melt.

The process of the present invention further comprises injecting a gas stream, for instance from a gas inlet line such as that shown at 22 in FIGS. 2 and 3, through the flow restrictor concurrently with metering the liquid composition into the flow restrictor, to create turbulent flow of gas at the outlet of the flow restrictor. The shear in the zone of turbulence atomizes the liquid composition.

The gas stream is controlled prior to injecting it through the flow restrictor. The gas stream may be heated by a heater, such as heater 24 as shown in FIG. 2. As noted above for the apparatus, when the liquid composition is a solution or slurry, the gas stream is heated to a temperature sufficient to vaporize the liquid of the solution or slurry and to leave the solid of the solution or slurry remaining. When the liquid composition is a melt, the gas stream should be heated to a temperature around the melt temperature of the liquid composition, to keep the liquid composition, and in particular, the melt, in liquid (i.e., melt) form. As also noted above for the apparatus, when using a melt, it is also helpful if auxiliary heat is provided to the first inlet line which supplies the melt prior to injection, to prevent pluggage of the line.

The process of the present invention also comprises the step of adding a particle to the zone of turbulence concurrently with the metering of the liquid composition and the injection of the gas stream. This mixes the particle with the atomized liquid composition at the zone of turbulence. This mixing at the zone of turbulence coats the particle with the liquid coating material. The particle is preferably metered in order to control the ratio of the particle and the liquid added at the zone of turbulence. This establishes the level of en coating of the particle. When a solution or slurry is used, the heat from the heated gas stream serves to evaporate the liquid of the solution or slurry, leaving the solid of the solution or slurry remaining to coat the particle. The mixing at the zone of turbulence then coats the particle with the remaining solid from the solution or slurry. When a melt is used, the mixing at the zone of turbulence coats the particle with the melt. Particle sizes should not exceed 20.0 mm.

As noted above, the zone of turbulence, which is the area of turbulent flow, is formed by the action of injecting the gas at high pressure through the flow restrictor. As discussed above with respect to the apparatus, it is preferable that the gas stream is accelerated to at least about one-half the velocity of sound prior to injection to ensure that a zone of turbulence of sufficient intensity will be formed at the outlet of the flow restrictor.

The residence time of the particles in the zone of turbulence is determined by the geometry of the first chamber and the amount of gas injected from the gas inlet line. The average residence time of the particle within the zone of turbulence is preferably less than 250 milliseconds. More preferably, the average residence time of the particle within the zone of turbulence is in the range of 25 to 250 milliseconds. Short residence times can be achieved because of the action of the zone of turbulence. The short residence times make the process of the present invention advantageous compared to conventional coating processes because the time, and hence, the cost of coating particles, are reduced. Typically, the particles are fed from a hopper, such as hopper 28 as shown in FIGS. 2 and 3, which is open to the atmosphere. As noted above for the apparatus, when the liquid composition is a melt, it is preferred that the particles be at ambient temperature because this will facilitate solidification of the melt after the melt (which is initially at a higher temperature) coats the particle in the zone of turbulence.

The process of the present invention may further comprise the step of adding another gas stream upstream of the zone of turbulence for cooling and conveying the coated particle. This other gas stream is added through a chamber, such as second chamber 32 as shown in FIGS. 2 and 3. As explained above for the apparatus, the pressure of the second gas stream must be sufficient to assist in conveying the coated particles from the zone of turbulence to the collection container, but should be at lower than the pressure of the first gas stream in order to achieve coating. When a solution or slurry is used, the solid of the solution or slurry cools and solidifies on the particle in the second chamber between the zone of turbulence and a collection container, such as collection zone 36 as described above. When a melt is used, the melt cools and solidifies on the particle in the second chamber between the zone of turbulence and the collection container. When a second chamber is not included, the solid or the melt cools and solidifies on the particle in the atmosphere between the zone of turbulence and the collection container, and the coated particles fall into the container.

The coating materials are generally liquid in nature, and can be single or multiple chemical compositions. Thus, they may be pure liquids, solutions, suspensions, emulsions, melted polymers, resins, and the like. These materials generally have viscosities in the 1 to 2,000 centipoise range. Coatings which are applied can be hydrophilic, hydrophobic or amphoteric in nature, depending on their chemical composition. When more than one coating is applied, it can be either as another shell adhering to the previous coating, or as individual particles on the surface of the material to be coated. These materials may also be reactive so that they cause the material they are coating to increase in viscosity or change to a solid or semi-solid material. So that the coating formed on the selected material is in the range stated above, the coating material should be capable of being molecularly dispersed, so that the coating can grow from the molecular level.

The apparatus as shown in FIGS. 2, 3, and 4 can be used for a number of processes. One such process is that of coating a soy product or flour with flavorings, colorants and the like. In this process, the soy product or flour enters the apparatus and the material that will be used to coat the soy product or flour is fed into the apparatus through the hopper into the high shear/turbulence zone. The resulting atomized coating material coats the surface of the soy product or flour, as it is pneumatically transported through the apparatus. The temperature of the process is at least 5° C. higher than vapor temperature of the solvent at the process operating pressure, so that the volatile materials in the coating mixture (e.g., water) are vaporized within a matter of milliseconds. The coated soy product or flour is then transported out of the apparatus in a substantially dry state, such that there is substantially no net moisture gain from one end of the process to the other. The net moisture gain is measured by a Cenco moisture balance operated at 105° C. Thus, the coating and drying of the material is accomplished in a single step. This is important so that the quality of the soy product or flour is maintained, as the particles are uniformly coated, the material is not degraded by excessive exposure to relatively high temperatures, and the particles do not agglomerate or stick to the sides of the vessels. Furthermore, the moisture level of the coated soy product or flour is substantially the same as the moisture level of the uncoated soy product or flour.

A convective drying process is used for removing residual volatiles that result from putting a solution, slurry or emulsion coating onto the surface of a particle. The design of the process precludes wet particles from reaching any wall to which they may stick, which improves the cleanliness of the system, and may also include a recycle system which can reduce any particle-to-particle or particle-to-wall sticking that might otherwise occur. This process may be selected from any number of methods, including but not limited to flash drying, pneumatic conveyor drying and spray drying, or combinations thereof. Residence times for drying are generally less than a minute, and preferably in the millisecond time frame.

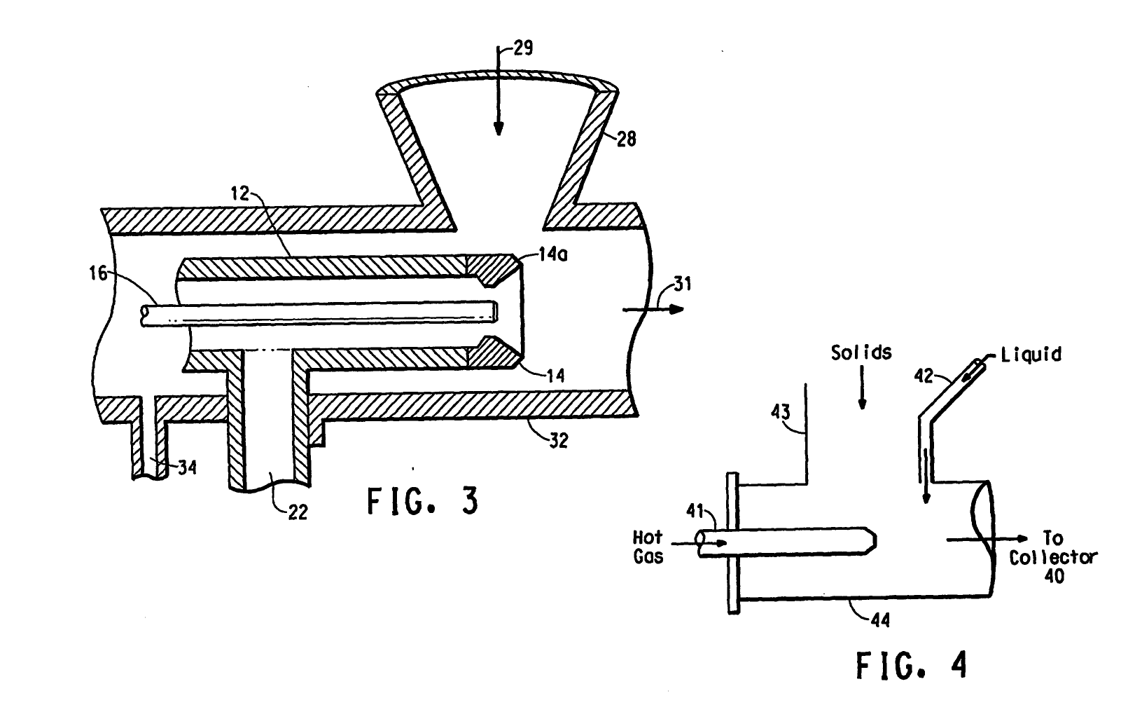

As shown in FIG. 4, the apparatus of FIGS. 2 and 3 can have an alternate configuration. Solids enter the apparatus through hopper 43. Liquid is added via a liquid inlet tube 42 located at the top of the apparatus, so that the liquid exits into the high shear/turbulence zone. Hot gas enters chamber 44 through nozzle 41. Product outlet from chamber 44 exits to collector 40. This configuration can allow for faster changes of liquid used for coating, and is less expensive to maintain.

### EXAMPLES

The invention is further described by the following Examples, which are provided for illustration and are not to be construed as limiting the scope of the invention.

Coating layers that were produced according to the Examples were calculated as their percent contribution to the mass of the coated particle. Coating levels were determined based on mass balance.

### Example 1

### Coating of Isolated Soy Protein Particles with Sucrose to Improve Dispersability

A preparation of isolated soy protein (“ISP”, Supro 500E, DuPont Protein Technologies, St. Louis, Mo.) was coated with a layer of sucrose in order to produce sucrosecoated protein particles in a single coating and drying process. The apparatus as shown in FIG. 4 had a mixing chamber 32 mm in diameter and 300 mm in length with a nozzle throat of 10 mm and a central liquid feed tube of 6.5 mm O.D. and 4.8 mm I.D. The apparatus has a single screw metering feeder (AccuRate) or a vibrating feeder (Syntron) for metering the solid particles. A peristaltic pump was fit with 6.5 mm Tygon elastomer tubing for metering the liquid. Supro 500E was used without further treatment and was metered to the system at 938 grams/minute. A solution of food grade sucrose (84% w/w in water) was metered at 94 g/min to the center tube using the peristaltic metering pump. Air was supplied to the nozzle at 345 KPa, and was at 316° C. at the nozzle. The air was used to atomize the sucrose solution, producing a negative pressure in the mixing zone to induce the addition of the Supro 500E, and to provide the heat for evaporating any residual moisture from the Supro 500E. The product of the mixing/drying was collected in a polyester twill bag filter immediately. The product had a coating of sucrose equal to 7.8% of the final mass of the coated particle. There was no gain in residual moisture (as measured by a Cenco moisture balance) when compared to the particles prior to coating. The sucrose-coated ISP retained the dry flowable property of the uncoated ISP starting material and possessed improved dispersability in liquid media.

Dispersability of particles was assessed on the basis of the completeness of dispersal of the coated and uncoated particles in water. Briefly, the method was as follows. Five grams of particles to be tested was added to 150 mL water in a beaker and the mixture was stirred rapidly for up to 20 s. The extent of dispersal was assessed through out the period of stirring. A sample of particles that was fully dispersed in 20 s was assigned a dispersal rating of 4.0, while a sample that dispersed in less than 20 s was assigned a rating of between 4 and 5, as indicated in Table 2. Samples of particles that were not fully dispersed after 20 s of stirring were assigned dispersability values in the range from 0 to 4 according to Table 2.

TABLE 2
Dispersability Ratings of Particles

| Stir time (s) | % Dispersion | Dispersability Rating |
|---|---|---|
| 20 | 0 | 0.0 |
| 20 | 25 | 1.0 |
| 20 | 50 | 2.0 |
| 20 | 56 | 2.3 |
| 20 | 63 | 2.5 |
| 20 | 69 | 2.8 |
| 20 | 75 | 3.0 |
| 20 | 81 | 3.3 |
| 20 | 88 | 3.5 |
| 20 | 94 | 3.8 |
| 20 | 100 | 4.0 |
| 16 | 100 | 4.3 |
| 12 | 100 | 4.5 |
| 8 | 100 | 4.8 |
| 5 | 100 | 5.0 |

The uncoated ISP that was used as the starting material for the coating process had a dispersability rating of 2.3. The particles that were coated with surcose to 7.8% of their weight had an improved dispersibility rating of 2.8.

### Examples 2-5

### Coating of Isolated Soy Protein Particles with Various Amounts of Sucrose to Improve Dispersability and to Provide a Barrier to Oxidation

Additional lots of sucrose-coated isolated soy protein particles were prepared using the apparatus and method of Example 1. Differing amounts of sucrose in the final product were achieved by modifying the operating parameters of the process. The modifications to the process and the amounts of sucrose in the products thus formed are listed in Table 3.

TABLE 3

| | Drying | Gas | | Sucrose | Sucrose | Sucrose in |
|---|---|---|---|---|---|---|
| | gas, | nozzle | ISP feed | feed | rate | finished |
| | pressure | temp. | rate | rate | temp. | particle |
| Example | (KPa) | (° C.) | (g/min) | (g/min) | (° C.) | (%) |
| 2 | Air, 345 | 331 | 11844 | 69 | 95 | 0.48 |
| 3 | Air, 345 | 329 | 923 | 80 | 95 | 6.8 |
| 4 | Air, 345 | 319 | 891 | 251 | 95 | 19.1 |
| 5 | Air, 345 | 319 | 554 | 195 | 95 | 22.8 |

The sucrose coatings that were layered onto the isolated soy protein particles proximately 0.5% to 23% of the final product. These Examples at a wide range of sucrose fractions can be deposited as a coating onto ISP particles by simple variation of the operating parameters of the process. The uncoated ISP that was used as the starting material for the coating process had a dispersability rating of 2.3. The particles that were coated with sucrose in these Examples had dispersibility ratings were measured to be as high as 3.8.

Demonstration of stabilization to oxidation provided by the sucrose coatings on the ISP particles was attempted by measurement of hexanal formed in the materials. Hexanal is formed by the oxidation of residual oil that is retained in isolated soy protein fractions. Hexanal content was measured by gas chromatography employing a flame ionization detector. Peak areas were obtained from integration of the detector trace from the analyses of samples that were uncoated and from samples that had been coated to various levels with sucrose. All samples were assayed after coating and after three weeks storage at 4.4° C. and at 43.3° C. The results are presented in Table 4.

TABLE 4
Hexanal formation in ISP with and without sucrose coatings

| | Hexanal content by FID (arbitrary units) | | |
|---|---|---|---|
| | | After storage for 3 weeks | |
| Material tested | Initial sample | At 4.4° C. | At 43.3° C. |
| Untreated ISP | 17200 | 17700 | 14500 |
| ISP coated in Ex. 2 | 16500 | 16100 | 14100 |
| ISP coated in Ex. 3 | 9810 | 13100 | 12900 |
| ISP coated in Ex. 4 | 10900 | 14900 | 17300 |
| ISP coated in Ex. 5 | 9520 | 13200 | 13200 |

The materials described above were subjected to testing by a trained sensory/odor panel to determine whether the coating of ISP with sucrose provided any benefit to development of off odors. The panel detected no differences in the odors of the materials irrespective of coating after three weeks storage at 4.4° C.

Although the results presented in Table 4, as well as those of the sensory panel were equivocal with respect to the ability of sucrose coating to suppress the development of undesired attributes in ISP, it is believed that incubation of the samples under more extreme or prolonged conditions will demonstrate differences in samples such as those described in the preceding Examples.

### Example 6

### Preparation of Isolated Soy Protein Particles Coated with Multiple Lavers of Sucrose to Improve Dispersability

The sucrose-coated isolated soy protein prepared in Example 1, above, was used as the solid feed material in the coating process of the invention to produce a particle with a multiple layers of sucrose. The apparatus was as described in Example 1 with the following operational modifications. The air that was used as the drying gas had a nozzle temperature of 320° C. The sucrose-coated isolated soy protein particles were metered into the apparatus at a rate of 1067 g/min. A solution of food grade sucrose (84% w/w in water) was metered into the apparatus at a rate of 91 g/min and at a temperature of 95° C. The dry coated particles were collected as described in Example 1. The resultant particle possessed a first, internal coating of sucrose and a second, external coating of sucrose that constituted 14.0% of the finished product. By repeated passage of coated isolated soy protein particles through the process of the invention it is possible to produce multiple coatings of varying amounts. The multiply coated particles had a dispersibility rating of 3.0.

### Examples 7-8

### Sucrose-Coated Isolated Soy Protein Particles Further Coated with a Layer of TiO₂ to Enhance Whiteness of the Particles

### Example 7

The sucrose-coated isolated soy protein prepared in Example 2, above, was used as the solid feed material in the coating process of the invention to produce a sucrose-coated particle with an outer layer of TiO₂. The apparatus was as described in Example 1 with the following operational modifications. The air that was used as the drying gas had a nozzle temperature of 315° C. The sucrose-coated isolated soy protein particles were metered into the apparatus at a rate of 828 g/min. A slurry of TiO₂ (72% wlw slurry of pigment grade material in water, DuPont, Wilmington, Del.) was metered into the apparatus at a rate of 49 g/min and at a temperature of 22° C. The dry coated particles were collected as described in Example 1. The resultant particle possessed a first, internal coating of sucrose and a second, external coating of TiO₂ constituting 4.1% of the finished product.

### Example 8

The sucrose-coated isolated soy protein prepared in Example 1, above, was used as the solid feed material in the coating process of the invention to produce a sucrose-coated particle with an outer layer of TiO₂. The apparatus was as described in Example 1 with the following operational modifications. The air that was used as the drying gas had a nozzle temperature of 302° C. The sucrose-coated isolated soy protein particles were metered into the apparatus at a rate of 884 g/min. A slurry of TiO₂ (72% w/w slurry of pigment grade material in water, DuPont, Wilmington, Del.) was metered into the apparatus at a rate of 87 g/min and at a temperature of 22° C. The dry coated particles were collected as described in Example 1. The resultant particle possessed a first, internal coating of sucrose and a second, external coating of TiO₂ constituting 6.6% of the finished product.

Whiteness of the particles was determined using a Hunter colorimeter (Hunter Associates Laboratory, Reston, Va.), as described in Hunter, R. S. (1952, “Photoelectric Tristimulus Colorimetry with Three Filters”, Circ. C. 429, U.S. Dept. Comm. Natl. Bur. Std. U.S.). A 5% w/w slurry of the particles was used after incubation at two different temperatures for three weeks. Two separate lots of theuncoated ISP were tested as control samples. The results of these determinations are presented in Table 5. It was concluded that coating of the particles with TiO₂ demonstrably improved the whiteness of the ISP particles.

TABLE 5
Whiteness Index Determinations

| | Whiteness Index | |
|---|---|---|
| TiO₂ Coating (% wt) | After storage at 43.3° C. | After storage at 4.4° C. |
| 0 | 39.9 | 41.1 |
| 0 | 39.3 | 40.0 |
| 4.1 (Example 7) | 42.1 | 42.3 |
| 6.6 (Example 8) | 48.8 | 49.8 |

As demonstrated by these examples, it is possible to coat particles with various amount of whitening agent by varying the operating parameters of the process.

### Examples 9-17

### Coating of Isolated Soy Protein Particles with Proteins to Provide Barriers to Oxidation

Many different preparations of isolated soy protein particles were produced that possessed an external layer of dried protein using the apparatus and method described in Example 1. The external protein layer served as a barrier to moisture and oxidation. The protein was applied as a solution that was metered into the apparatus with a peristaltic pump. Three different protein materials were used as coatings. Gelatin (Leiner Davis Gelatin, West Chester, Pa.) was provided as a 26% w/w aqueous solution for the materials produced in Examples 9, 10, and 11. Zein (F400 corn zein, Freeman Industries, Tuckahoe, N.Y.) was provided as a 20% solution in 90% EtOH/10% water, and was used in preparing the materials in

Examples 12 and 13. Casein (Non Fat Dry Milk Powder Low Heat A Grade, T. C. Jacoby & Co. Inc., St Louis, Mo.) was provided as a 20% aqueous solution for the materials produced in Examples 14-17. Supro 500E was used without further treatment in Examples 9-13. Supro 670E (DuPont Protein Technologies, St. Louis, Mo.) was used without further treatment in Examples 14-17. The apparatus and the collection of the dry coated particles were as described in Example 1. By modification of the operating parameters of the process, particles were produced that possessed differing quantities of protein as a barrier layer. The modifications to the process and the amounts of protein that were delivered as external coating are listed in Table 6.

TABLE 6

| | | | | | | Protein |
|---|---|---|---|---|---|---|
| | Drying | Gas | ISP | Protein | Protein | coating in |
| | gas, | nozzle | feed | feed | feed | finished |
| | pressure | temp. | rate | rate | temp. | particle |
| Example | (Kpa) | (° C.) | (g/min) | (g/min) | (° C.) | (%) |
| 9 | Air, 345 | 350 | 605 | 176 | 80 | 5.5 |
| 10 | Air, 345 | 350 | 1483 | 198 | 80 | 2.6 |
| 11 | Air, 345 | 353 | 1524 | 232 | 80 | 3.0 |
| 12 | Air, 345 | 272 | 515 | 334 | 30 | 9.4 |
| 13 | Air, 345 | 315 | 750 | 343 | 30 | 6.8 |
| 14 | Air, 448 | 270 | 155 | 11 | 30 | 1.4 |
| 15 | Air, 448 | 233 | 150 | 25 | 30 | 3.2 |
| 16 | Air, 448 | 249 | 270 | 40 | 30 | 2.9 |
| 17 | Air, 448 | 279 | 207 | 75 | 30 | 5.3 |

These Examples demonstrate that it is possible to apply various quantities of a protein as an external coating onto isolated soy protein to create particles with oxidation-moisture barrier layers of varying thickness and composition.

It is believed that coatings of ISP particles with proteins, as described in the preceding Examples, will provide protection of the residual oil in ISP from oxidative degradation.

### Examples 18-28

### Coating of Isolated Soy Protein Particles with Lipids

A number of Different Preparations of Lipid-Coated Isolated Soy Protein particles were produced using the apparatus and method described in Example 1.

The external lipid layer served as either an aid to dispersability or as a barrier to moisture. The lipid was applied as a pure liquid that was metered into the apparatus with a peristaltic pump. Supro 500E was used without further treatment in Examples 18-28. Three different lipid materials were used as coatings. Lecithin (Metarian DA51, Degussa Texturant Systems, Freising, Germany) was used in production of the materials in Examples 18-23. DURKEX (a high stability vegatable oil from Loders Croklaan, Wormerveer, Netherlands) was used in production of the materials in Examples 24-26. Dritex (a high melting temperature fat from ACH Food Companies, Cordova, Tenn.) was used in production of the materials in Examples 27 and 28. The collection of the dry coated particles were as described in Example 1. By modification of the operating parameters of the process, particles were produced that possessed differing quantities of lipid as a barrier layer. The modifications to the process and the amounts of lipid that were delivered as external coating are listed in Table 7.

TABLE 7

| | | | | | | Lipid |
|---|---|---|---|---|---|---|
| | Drying | Gas | ISP | Lipid | Lipid | coating in |
| | gas, | nozzle | feed | feed | feed | finished |
| | pressure | temp. | rate | rate | temp. | particle |
| Example | (KPa) | (° C.) | (g/min) | (g/min) | (° C.) | (%) |
| 18 | Air, 448 | 22 | 394 | 3.7 | 30 | 0.9 |
| 19 | Air, 448 | 22 | 829 | 10.8 | 30 | 1.3 |
| 20 | Air, 448 | 22 | 753 | 7.8 | 30 | 1.0 |
| 21 | Air, 448 | 22 | 314 | 6.0 | 30 | 1.9 |
| 22 | Air, 448 | 22 | 632 | 8.0 | 30 | 1.3 |
| 23 | Air, 448 | 22 | 652 | 7.0 | 30 | 1.1 |
| 24 | Air, 448 | 22 | 992 | 7.8 | 30 | 0.8 |
| 25 | Air, 448 | 22 | 153 | 5.8 | 30 | 3.6 |
| 26 | Air, 448 | 22 | 1471 | 8.0 | 30 | 0.5 |
| 27 | Air, 345 | 142 | 550 | 125 | 80 | 18.5 |
| 28 | Air, 345 | 124 | 556 | 137 | 80 | 19.8 |

These Examples demonstrate that it is possible to apply various quantities of a lipid as an external coating onto isolated soy protein particles.

The particles coated with lecithin or DURKEX were tested for improved disperability compared to the coated ISP starting material. Dispersability was determined as described above for the sucrose-coated ISP particles in Examples 1-5. The results of the dispersibility mesurements for the lipid coated particles are presented in Table 8.

TABLE 8
Dispersability ratings for lipid-coated ISP

| | Lipid | Coating on particles | Dispersability |
|---|---|---|---|
| Material tested | coating | (%) | rating |
| Uncoated ISP | N/A | N/A | 2.3 |
| Material from Ex. 18 | Lecithin | 0.9 | 3.6 |
| Material from Ex. 19 | Lecithin | 1.3 | 3.9 |
| Material from Ex. 21 | Lecithin | 1.9 | 4.1 |
| Uncoated ISP | N/A | N/A | 2.0 |
| Material from Ex. 24 | DURKEX | 0.8 | 3.5 |
| Material from Ex. 25 | DURKEX | 3.6 | 4.1 |
| Material from Ex. 26 | DURKEX | 0.5 | 3.3 |

The results reported in Table 8 demonstrate that dispersability improved with coating of ISP particles with either lecithin or DURKEX.

The high-melting temperature fat that was used to coat the ISP particles produced in Examples 27 and 28 provided protection of the ISP against moisture. The effectiveness of the moisture barrier was demonstrated by differential, temperature-dependent dispersal of the protein into water. High melting point fat-coated particles (approximately 1 g) were placed into 150 mL of water in a beaker at room temperature. A portion of the fat-coated particles floated on the surface of the water, while the remainder sank below the surface. The beaker was shaken gently and observed for 5 minutes. No substantial change in the appearance of the water was observed. It was concluded that the soy protein in the particles were protected from the water by the fat barrier because the water did not become cloudy. Similarly, high melting point fat-coated particles were added to water at 90° C. Within a few seconds, the particles dispersed into the water and the water became cloudy and off-white. It was concluded that the protection of the soy protein from the water by fat was not possible above the melting point of the fat barrier (70° C.). Thus the protein was delivered to the water at a temperature that caused the fat to melt.

### Example 29

### Coating of Cereal Flour with Red Pigment

Wheat flour (Gold Medal All Purpose, General Mills, Inc., Minneapolis, Minn.) was coated with red dye in order to produce a red colored flour that is suitable for preparing colored baked goods. The apparatus and process was as described in Example 1 with the following operating modifications. Nitrogen was used as the drying gas and was heated 300° C. upstream of the nozzle. The flour particles were metered into the apparatus at a rate of 500 g/min. A solution of red dye was prepared by dissolving “Cardinal Red” Rit® dye (Unilever Besffoods, North America, Englewood Cliffs, N.J.) to a red dye concentration of 30% w/w in water. The dye solution was metered into the apparatus at a rate of 25 g/min and at a temperature of 22° C. The dry coated particles were collected in a single bag dust collector. The colored flour was a bright red material with a particle size distribution and moisture content indistinguishable from the starting material.

### Example 30

### Coating of Cereal Flour with Blue Pigment

Wheat flour (Gold Medal All Purpose, General Mills, Inc., Minneapolis, Minn.) was coated with blue dye in order to produce a blue colored flour that is suitable for preparing colored baked goods. The apparatus and process was as described in Example 1 with the following operating modifications. Nitrogen was used as the drying gas and was heated 300° C. upstream of the nozzle. The flour particles were metered into the apparatus at a rate of 500 g/min. A solution of blue dye was prepared by dissolving “Denim Blue” Rit® dye (Unilever Bestfoods, North America, Englewood Cliffs, N.J.) to a concentration of 30% w/w in water. The dye solution was metered into the apparatus at a rate of 25 g/min and at a temperature of 22° C. The dry coated particles were collected in a single bag dust collector. The flour was a bright blue material with a particle size distribution and moisture content indistinguishable from the starting material.

### Example 31

### Coating of Soy Flour with Sucrose to Improve Dispersability

Soy flour (DuPont Protein Technologies, St. Louis, Mo.) was coated with sucrose in order to improve dispersability. The apparatus and process was as described in Example 1 with the following operating modifications. Air was used as the drying gas and was heated 239° C. upstream of the nozzle. The soy flour particles were metered into the apparatus at a rate of 950 g/min. A solution of food grade sucrose (84% w/w in water) was metered into the apparatus at a rate of 114 g/min and at a temperature of 95° C. The dry coated particles were collected in a single bag dust collector. The coated flour retained the dry flowable property of the uncoated soy flour starting material and possessed improved dispersability in water when measured according to the method described in Example 1.

1. A process for coating a soy product, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating, wherein the gas stream is optionally heated; and
(c) adding a soy product to the region of turbulent flow concurrently with steps (a) and (b), wherein the soy product mixes with the atomized liquid coating material to provide a coated soy product.

2. The process of claim 1 wherein the soy product is selected from the group consisting of soy protein isolate, soy concentrate, soy meal, soy cotyledon fiber, dehulled soybeans, soy hypocotyls, soy grits, soy chips, soy flour, textured soy protein, and soy flakes,

3. The process of claim 1 wherein the liquid coating material is selected from the group consisting of a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, an emulsification agent or a mixture thereof.

4. The process of claim 1 further comprising repeating steps (a)-(c) at least once wherein the liquid coating material is the same or different.

5. A coated soy product made by the process of any of claims 1-4.

6. A food comprising a coated soy product made by the process of any of claims 1-4.

7. A nutritional supplement comprising a coated soy product made by the process of any of claims 1-4.

8. A beverage comprising a coated soy product made by the process of any of claims 1-4.

9. Infant formula comprising a coated soy product by the process of any of claims 1-4.

10. A pet food comprising a coated soy product by the process of any of claims 1-4.

11. Animal feed comprising a coated soy product by the process of any of claims 1-4.

12. Use of a coated soy product made by the process of any of claims 1-4 as a food ingredient, a nutritional supplement ingredient, a beverage ingredient, an infant formula ingredient, a pet food ingredient or an animal feed ingredient.

13. A process for coating a flour, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating material, wherein the gas stream is optionally heated; and
(c) adding a flour to the region of turbulent flow concurrently with steps (a) and (b), wherein the flour mixes with the atomized liquid coating material to provide a coated flour.

14. The process of claim 13 wherein the flour is selected from the group consisting of soy flour, wheat flour, oat flour, rye flour, barley flour, rice flour, millet flour, corn flour and filler flour.

15. The process of claim 13 wherein the liquid coating material is selected from the group consisting of a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, an emulsification agent or a mixture thereof.

16. The process of claim 13 further comprising repeating steps (a)-(c) at least once wherein the liquid coating material is the same or different.

17. A coated flour made by the process of any of claims 13-16.

18. A food comprising a coated flour made by the process of any of claims 13-16.

19. A baked good comprising a coated flour made by the process of any of claims 13-16.

20. A snack food a coated flour made by the process of any of claims 13-16.

21. Use of a coated flour made by the process of any of claims 13-16 in as a food ingredient.
